SEQUENCE LISTING (16 sequences)

SEQ ID NO: 1 - DNA, 21 bp, Artificial Sequence
  Description of Artificial Sequence Synthetic probe
gctcattgtgggtccagtctt

SEQ ID NO: 2 - DNA, 20 bp, Artificial Sequence
  Description of Artificial Sequence Synthetic probe
ggatgggcagtagaattttg

SEQ ID NO: 3 - DNA, 21 bp, Artificial Sequence
  Description of Artificial Sequence Synthetic probe
actgatcacccagctagaaag

SEQ ID NO: 4 - DNA, 21 bp, Artificial Sequence
  Description of Artificial Sequence Synthetic probe
tggtcaagcaaacgtttaagt

SEQ ID NO: 5 - DNA, 21 bp, Artificial Sequence
  Description of Artificial Sequence Synthetic probe
cttgcaacttgtctggcccac

SEQ ID NO: 6 - DNA, 21 bp, Artificial Sequence
  Description of Artificial Sequence Synthetic probe
gactctaatacgctggtcaag

SEQ ID NO: 7 - DNA, 21 bp, Artificial Sequence
  Description of Artificial Sequence Synthetic probe
aaaacaggtaaatgtggtatg

SEQ ID NO: 8 - DNA, 21 bp, Artificial Sequence
  Description of Artificial Sequence Synthetic probe
taagttctctcgcccactgtg

SEQ ID NO: 9 - DNA, 21 bp, Artificial Sequence
  Description of Artificial Sequence Synthetic primer
cagacgacttgaatgggaacg

SEQ ID NO: 10 - DNA, 19 bp, Artificial Sequence
  Description of Artificial Sequence Synthetic primer
atgcatagaaagctgttgg

SEQ ID NO: 11 - DNA, 21 bp, Artificial Sequence
  Description of Artificial Sequence Synthetic probe
taacttctcgcgcccactgtg

SEQ ID NO: 12 - DNA, 21 bp, Artificial Sequence
  Description of Artificial Sequence Synthetic probe
aaaatgggtaaatgtggtatg

SEQ ID NO: 13 - DNA, 21 bp, Artificial Sequence
  Description of Artificial Sequence Synthetic probe
gactctaataggctggtcaag

SEQ ID NO: 14 - DNA, 21 bp, Artificial Sequence
  Description of Artificial Sequence Synthetic probe
gattctaataggcttgtcaag

SEQ ID NO: 15 - DNA, 21 bp, Artificial Sequence
  Description of Artificial Sequence Synthetic probe
gattctaatagacttgtcaag

SEQ ID NO: 16 - DNA, 21 bp, Artificial Sequence
  Description of Artificial Sequence Synthetic probe
ggctctaataggctggtcaag

(19) United States
(12) Patent Application Publication
(10) Pub. No.: US 2006/0275809 A1
(43) Pub. Date: Dec. 7, 2006

(54) Rapid identification of the varieties and genotypes of cryptococcus neoformans species complex using a high-throughput flow cytometer

(75) Inventors: Mara R. Diaz, Key Biscayne, FL (US); Jack W. Fell, Miami, FL (US)

Correspondence Address: VENABLE LLP, P.O. BOX 34385, WASHINGTON, DC, 20043-9998, US

(73) Assignee: University of Miami, Miami, FL (US)

(21) Appl. No.: 11/435,291

(22) Filed: May 17, 2006

Related U.S. Application Data
(60) Provisional application No. 60/681,480, filed on May 17, 2005.

(51) Int. Cl. C12Q 1/68; C07H 21/04; C12M 1/34

(52) U.S. Cl. 435/6; 435/287.2; 536/24.1

(57) ABSTRACT

Nucleic acid probes and molecular method to identify the varieties and genotypic groups within C. neoformans species complex. The method employs a flow cytometer with a dual laser system that allows the simultaneous detection of different target sequences in a multiplex and high-throughput format. The assay uses a liquid suspension hybridization format with specific oligonucleotide probes that are covalently bound to the surface of fluorescent color-coded microspheres. Biotinylated target amplicons, which hybridized to their complementary probe sequences, are quantified by the addition of the conjugate, streptavidin-R-phycoerythrin. The assay is specific and sensitive, and allows discrimination of 1 bp mismatch with no apparent cross-reactivity and is capable of detecting 10¹ to 10³ genome copies. The assay can be used directly with yeast cells or isolated DNA, can be undertaken in less than one hour following PCR amplification and permits identification of species in a multiplex format. In addition, to multiplex capability, the assay allows simultaneous detection of target sequences in a single reaction.

### CROSS-REFERENCE TO RELATED APPLICATION

This application claims priority to U.S. provisional application No. 60/681,480, filed May 17, 2005, which is incorporated herein by reference in its entirety.

This research was funded by National Institute of Health Grant 1-UO1 AI53879-01. The U.S. Government has certain rights in the invention.

### BACKGROUND OF THE INVENTION

1. Field of the Invention

The invention relates to species-specific nucleic acid probes and a method for using the probes to detect cryptococcosis infection.

2. Background Information

Cryptococcosis, caused by the basidiomycetous yeast Cryptococcus neoformans (Sanfelice) Vuillemin, is a disease that has gained a great deal of attention in Europe, America, Africa and Southeast Asian countries (3, 22, 60, 63, 73, 74). Prior to highly active anti-retroviral treatment (HAART), cryptococcosis was considered the fourth most common cause of mortality in AIDS individuals (45). In recent years, the incidence of cryptococcosis in America and Europe has decreased but it continues to be a serious and fatal disease in immunosuppressed HIV individuals who have limited access to HIV medical care (60). In Africa, Cryptococcus neoformans ranks as the most common life threatening fungal pathogen in AIDS (25) with mortality rates as high as 100% in young adults in a teaching hospital in Zambia (61). A survey conducted during 1992 and 2000 in four major southern metropolitan areas in the United States, showed that 89% of the individuals who contracted cryptococcosis carried the HIV virus (60). C. neoformans has strong predilection for the meninges and the spinal fluid in AIDS patients. Cryptococcal pneumonia is common in non-AIDS patients, especially for those who undergo chemotherapy or organ transplantation (38). Also, mortality rates as high as 81% have been documented in the USA with patients suffering from cirrhosis (75).

The encapsulated yeast C. neoformans represents a species complex comprising two species: C. neoformans var. grubii (serotype A) and var neoformans (serotype D), and C. gattii (serotypes B and C). In addition, there is a hybrid serotype AD. The varieties differ in their geographical distribution, ecology, physiology, and their molecular and morphological characteristics (8, 12, 20, 43). Although C. neoformans var. grubii ranks as the most common cause of cryptococcal meningitis, there are reports that indicate an increase in the incidence of C. neoformans var. neoformans (seroptype D) isolates (77). In addition, serotype D clinical strains are frequently encountered in Europe (23, 47). C. gattii, which commonly infects patients with normal immune systems, appears to occur in tropical and subtropical areas as opposed to the cosmopolitan worldwide distribution of C. neoformans var. grubii and C. neoformans var. neoformans (5, 28). However, the geographic boundary expanded with the recent outbreak of C. gattii in the Vancouver Islands, British Columbia (44, 78). This unprecedented outbreak involved 50 individuals as well as terrestrial animals and marine mammals (78).

Classical yeast identification techniques are often used for the diagnosis of cryptococcosis. These methods are based on physiological, histopathological, biochemical and morphological analyses. Some of these tests are elaborate and can lead to problems in accurate identification resulting in erroneous identification, diagnosis and treatments. Culture techniques employing selective isolation media such as niger seed or dopamines are often used for the identification of C. neoformans species complex, but this method relies on the ability of the strain to grow and can be time consuming, which can result in delay of treatment. The india ink direct examination is another common diagnostic test employed in many clinics. Although easy to perform, this test identifies only 50% of cryptococcosis cases in non-AIDS and 80% in AIDS patients (45). The specificity of this test can be reduced by the presence of leukocytes, myelin globules, fat droplets and tissue cells (12). The urease test, which is based on the ability of the strains to hydrolyze urea can take up to four days and is not a discriminatory test since all basidiomycetous yeasts can hydrolyze urea and a few strains of the species have been reported to be negative (4). Serological diagnostic tests, such as MYCO-Immune (American Micro Scan, NJ); CALAS (Meridian Diagnostics Inc., OH) and IMMY (Immuno-mycologics, OK) are 95% sensitive and specific but often lead to false positive and negative results (7, 9, 36, 59).

Molecular techniques have been used for the identification of C. neoformans species complex, some of which include: RAPD (43); AFLP (8); karyotyping (11, 66); PCR fingerprinting (16, 27, 43, 58); sequencing (20, 43) and PCR-RFLP (23, 48). Even though these techniques have been successful at identifying C. neoformans at species and genotypic level, some of these techniques are cumbersome and not easily adapted for use in routine diagnostic laboratories (48). The present study describes a rapid and reliable molecular bead-based method that allows the simultaneous detection of the varieties and genotypes of C. neoformans species complex. This molecular assay uses specific oligonucleotide probes derived from unique sequence areas of the intergenic spacer (IGS) region of ribosomal DNA. Based on sequence divergences in the IGS region, which is a non-conservative, fast evolving region frequently used as a tool for species identification (20, 21, 30, 69), Diaz et al. (2000) showed that C. neoformans, portrayed five distinct phylogenetic lineages represented by genotype 1 with sub-genotypes 1a, 1b, 1c (Cryptococcus neoformans var. grubii); genotype 2 with sub-genotypes 2a, 2b, 2c (Cryptococcus neoformans var. neoformans); and genotypes 3, 4 and 5, represented by C. gattii. Recently, a new IGS genotypic group comprised of one isolate from Africa and two from India was found (unpublished). Therefore, this new genotypic group, which is phylogenetically closely related to genotypes 4 and 5 within the C. gattii complex, has been added to our list as genotype 6.

### SUMMARY OF THE INVENTION

The invention described herein includes, inter alia, a rapid, sensitive, and specific molecular assay with high throughput capability to identify the varieties and genotypic groups of the species complex of Cryptococcus neoformans. These variants include, but are not limited to, var. grubii (serotype A), var. neoformans (serotype D), C. gattii (serotypes B and C) and the genotypes comprising C. neoformans species complex. In a preferred embodiment, this method uses Luminex xMap® technology, a flow cytometer that allows the simultaneous identification of the varieties using microsphere sets that contain specific capture probes derived from target sequences. Capture probes that have been found to be particularly useful are

| | |
|---|---|
| GCTCATTGTGGGTCCAGTCTT, | (SEQ ID NO: 1) |
| GGATGGGCAGTAGAATTTTG, | (SEQ ID NO: 2) |
| ACTGATCACCCAGCTAGAAAG, | (SEQ ID NO: 3) |
| TGGTCAAGCAAACGTTTAAGT, | (SEQ ID NO: 4) |
| CTTGCAACTTGTCTGGCCCAC, | (SEQ ID NO: 5) |
| GACTCTAATACGCTGGTCAAG, | (SEQ ID NO: 6) |
| AAAACAGGTAAATGTGGTATG, | (SEQ ID NO: 7) |
| and | |
| TAAGTTCTCTCGCCCACTGTG. | (SEQ ID NO: 8) |

The disclosed molecular test uses Luminex xMAP technology, however, it should be evident to those of skill in the art that the probes of the invention may be useful in any hybridization-based assay. The Luminex xMAP technology is a flow cytometer technology that employs 5.6 μm polystyrene carboxylated microspheres that permits the simultaneous detection of 100 analytes by combining 100 different combinations of microspheres in a single reaction. Each microsphere set is internally dyed with different intensities of two spectral fluorochromes, and their unique spectral emission is recognized by a red laser. Specific oligonucleotide sequences, which are complementary to the target sequence, are covalently bound to unique sets of fluorescence beads. Upon hybridization, the biotinylated amplicon bound to the surface of the microsphere is recognized by a green laser that quantifies the fluorescence of the reporter molecule (streptavidin-R-phycoerythrin) (32). By adding a reporter molecule (streptavidin R-phycoerythrin) all hybridized species-specific amplicons captured by their complementary nucleotide sequence in the microsphere beads are recognized by the fluorescence of the reporter molecule. The median fluorescent intensity (MFI) of the reporter molecule is then used to quantify the amount of DNA bound to the beads.

As used herein, the term “amplicon” refers to DNA that has been synthesized using amplification techniques such as PCR or LCR. However, DNA to be tested according to the methods of the invention need not be the product of any particular process. Other types of nucleic acid, e.g. RNA, may also be tested using the compositions and methods of the invention, and capture probes of the invention may also be comprised, for example, of RNA.

The Luminex xMAP technology is based on polystyrene beads (microspheres) that are internally dyed with two spectrally distinct fluorescent dyes. Using precise concentrations of these fluorescent dyes, an array consisting of 100 distinct sets microspheres are color coded. Each set can carry a different reactant on its surface. Since individual beads can be distinguished by their spectral address, once the sets are combined combined, up to 100 different analytes can be measured simultaneously in a single reaction vessel.

Each such bead within the set is said to have a specific spectral address.

This technology has been adapted to a wide variety of applications involving human single nucleotide polymorphisms (SNPs) (84), bacterial identification (24, 83, 85), Y chromosome SNPs analysis (81), and kinase assays for drug discovery (82).

Accordingly, the invention includes capture probes useful for the detection and identification of fungal infections, in particular for the identification of species within the genus Cryptococcus. The capture probes of the invention will generally comprise oligonucleotides of 15-25 bases in length, preferably 20-22 bases, but may be larger or smaller. Oligonucleotides of 16, 17 and 18 bases in length are also considered to be particularly useful. Examples of preferred capture probes of the invention are presented in Table 2. The invention also includes probes whose sequences are complementary to those presented in Table 2. The capture probes themselves may comprise, consist essentially of, or consist of these oligonucleotides. Fragments of the listed probes and complementary probes are also expected to be useful.

TABLE 2

Probes sequences used for the detection of the
varieties and genotypic groups of the species
complex, Cryptococcus neoformans

| Probe | Sequence | Target |
|---|---|---|
| CNN b | GCTCATTGTGGGTCCAGTCTT | C. n. var. grubii/C. |
| | (SEQ ID NO: 1) | n. var. neofformans |
| | | (genotypes 1-2) |
| CNN 1b | GGATGGGCAGTAGAATTTTG | C. n. var. grubii |
| | (SEQ ID NO: 2) | (genotype 1) |
| CNN 2d | ACTGATCACCCAGCTAGAAAG | C. n. var. neoformans |
| | (SEQ ID NO: 3) | (genotype 2) |
| CNG | TGGTCAAGCAAACGTTTAAGT | C. n. gattii |
| | (SEQ ID NO: 4) | (genotypes 3-4-5-6) |
| CNG 3 | CTTGCAACTTGTCTGGCCCAC | C. n. gattii |
| | (SEQ ID NO: 5) | (genotype 3) |
| CNG 4c | GACTCTAATACGCTGGTCAAG | C. n. gattii |
| | (SEQ ID NO: 6) | (genotype 4) |
| CNG 5b | AAAACAGGTAAATGTGGTATG | C. n. gattii |
| | (SEQ ID NO: 7) | (genotype 5) |
| CNG 6 | TAAGTTCTCTCGCCCACTGTG | C. n.. gattii |
| | (SEQ ID NO: 8) | (genotype 6) |

Although the capture probes of the invention may be used in solution, they are particularly useful when bound to solid supports. In a preferred embodiment, the capture probes will be labeled with a detectable label, for example, a radioactive or fluorescent label. In one particularly preferred embodiment, the probes are bound to fluorescent beads to allow separation and identification of bound products. The capture probes may also be bound to a solid support, such as a multiwell plate or a solid matrix to form a microarray. Solid phases or solid supports include, but are not limited to those made of plastics, resins, polysaccharides, silica or silica-based materials, functionalized glass, modified silicon, carbon, metals, inorganic glasses, membranes, nylon, natural fibers such as silk, wool and cotton, and polymers, as will be know to those of skill in the art.

Examples of useful arrays include an array of color-coded beads (Luminex; Austin, Tex.), an array of radiofrequency-tagged beads (PharmaSeq; Monmouth Junction, N.J.), an array of nanocrystal encoded beads (Quantum Dot, Hayward, Ca.), an array of radioisotopically labeled beads, or a three dimensional microarray. Thus, the location of each probe on the solid phase microarray enables the identification of each target species that is bound.

The sequences/probes of the invention may be used singly, but also may be advantageously used in combination with other sequences/probes of the invention, for example in combinations of 2, 3, 4, 5, 6, 7, 8, etc., up to an including all of the probes described herein. The probes may also be used in combination with other probes, e.g. probes from other pathogens, for example, for diagnosis of infection.

It is also an object of the invention to provide a method for detecting fungal pathogens, particularly yeast pathogens, using the capture probes of the invention. In one embodiment, the method comprises the steps of obtaining a set of fluorescent beads covalently bound to species-specific capture probes; contacting said fluorescent beads with a biological sample that may contain species for which said capture probes are specific under conditions such that the target species will bind to the capture probes; using a first laser to classify the target species/probes complexes by their spectral addresses; and quantitating the complexes using fluorescent detection. Useful variations of this method will be apparent to those of skill in the art. In a particularly preferred embodiment, the capture probe is specific for at least one species/strain of the genus Cryptococcus. Examples of suitable capture probes are shown in Table 2. Complements of these probes and equivalent or corresponding RNA sequences will also be useful. By “complement” is meant any nucleic acid that is completely complementary over the entire length of the sequence, as understood in the art.

In one embodiment, a method is provided for detecting a fungal pathogen comprising the steps of providing at least one capture probe comprising a DNA sequence selected from Table 2, a complement thereof, or a corresponding RNA sequence; contacting the capture probe(s) with a biological sample that may contain target species of nucleic acid for which said capture probe(s) are specific under conditions such that the target species will become bound to the probe to produce a hybridized product; and detecting the presence or absence of hybridized product, the presence of said hybridized product being indicative of the presence of said fungal pathogen. The method may further comprise the step of measuring or quantitating any hybridized product that is detected. The capture probe may bound to a solid support, as described above.

In another embodiment, a method is provided for detecting fungal pathogens comprising the steps of obtaining a set of fluorescent beads covalently bound to capture probes; contacting the fluorescent beads with a biological sample that may contain amplicons of target species for which the capture probes are specific under conditions such that the amplicons will become bound to the probe to produce a hybridized product; using a first laser to classify the beads by their spectral addresses; and detecting the presence or absence of said hybridized product, the presence of said hybridized product being indicative of the presence of said fungal pathogen. The method may further comprising the step of quantitating hybridized biotinylated amplicons using fluorescent detection. In a preferred embodiment, the first laser has a wavelength of 635 nm. In another preferred embodiment, the hybridized biotinylated amplicons are quantified with a 532 nm laser. In a particularly preferred embodiment, the capture probe is specific for a species or strain from the genus Cryptococcus, particularly for a strain of C. neoformans or C. gattii. Capture probes comprising the sequences of Table 2, complements thereof, or corresponding RNA sequences are considered to be especially useful.

Also provided is a kit (e.g. a diagnostic kit) comprising at least one capture probe as described above, optionally including instructions for use. The kit will usually include a plurality of such capture probes, for example, at least 2, 3, 4, 5, 6, 7 or 8 of said capture probes. The kit may also include capture probes for other infectious microorganisms, e.g. for differential diagnosis.

### BRIEF DESCRIPTION OF THE DRAWINGS

FIG. 1: IGS phylogenetic tree of C. neoformans species complex (heuristic search, random stepwise addition (PAUP 4.0b10) based on sequence analysis of the intergenic spacer rDNA (˜800 bp).

FIG. 2: MFI of CNG 4c probe tested with strains representing all genotypic groups within C. neoformans species complex. Nucleotide variations from the probe sequence are depicted in bold lower case. Sequences are identified as SEQ ID NOS: 6 and 13-16, in order of appearance.

FIG. 3A-3H: Probe response with target and non-taret DNA. The hybridization was performed at 55° C. with amplicons biotinylated at the 5′ end. All probes were tested in a multiplex format (eight-plex assay). The background signal was substracted. A. CNNb (genotypes 1 and 2); B. CNN 1b (genotype 1); C. CNN2d (genotype 2); D. CNG (genotypes 3-4-5-6); E. CNG 3 (genotype 3); F. CNG 4c (genotype 4); G. CNG 5c (genotype 5) and CNG 6 (genotype 6). Values are mean fluorescence intensity (MFI).

FIG. 4: MFI of probes CNG 4c, CNG, and CNN 1b tested in uni-plex and eight-plex format.

FIG. 5: MFI of probes tested with 5 and 10 ng of genomic DNA from 5 strains representing 5 different genotypes.

FIG. 6: Effect of various amount of amplicon template mix on hybridization intensities. Amplicon products were derived from a simultaneous PCR amplification of five different DNA targets: WM 554, CBS 7523, CGBMA6, CBS 6955 and CBS132. The PCR reaction used 5 ng of each of the above targets. Comparison in signals between single target PCR (one strain) vs multi-target PCR (5 strains) is provided.

FIG. 7: Signal at various concentrations of genomic DNA.

FIG. 8: Direct amplification and detection of DNA targets. After hybridization, 5 μl of the PCR product was tested with its complementary probe sequence. The hybridization assay was performed in an eight-plex assay. Samples were run in duplicate and the experiment was run twice. Values are given as mean fluorescence intensity.

### DETAILED DESCRIPTION

Materials and Methods

Isolates

Clinical and environmental DNA isolates from different geographic areas were analyzed. The source of isolation, genotypes, and serotypes are described in Table 1. Serotype data was obtained from CBS collection, Boekhout et al. (2001) or by information provided by the depositors of the isolates.

TABLE 1
List of experimental strains used to develop the probes and their source of isolation,
serotype and IGS genotype.

| Strain | Source of Isolation | Serotype | Genotype |
|---|---|---|---|
| C. neoformans | | | |
| var. grubii | | | |
| AVB12 RDA 4054 | AIDS patient, The Netherlands | A | 1 |
| CBS 916 | Unknown | A | 1 |
| Hamdan 214L | AIDS patient, Brazil | A | 1 |
| Hamdan MCP2 | Pigeon dropping, Brazil | A | 1 |
| RV 58146 | Wood, Zaire | A | 1 |
| RV 59351 | Parrot droppping, Belgium | A | 1 |
| RV 62210 | Cerebrospinal fluid from AIDS patient, Belgium | A | 1 |
| WM 148 | Human, cerebrospinal fluid, Australia | A | 1 |
| WM 553 | House dust, Brazil | A | 1 |
| WM 554 | Dust from pigeon, Brazil | A | 1 |
| WM 626 | Human cerebrospinal fluid, Australia | A | 1 |
| WM 712 | Cat paranasal, Australia | A | 1 |
| WM 719 | AIDS patient, India | A | 1 |
| WM 721 | Pigeon dropping, India | A | 1 |
| WM 723 | Environmental isolate, USA | A | 1 |
| NIH 192 | Desert soil, USA | A | 1 |
| NIH 193 | Soil, USA | A | 1 |
| NIH 443 | Soil, USA | A | 1 |
| H 99 | Patient with Hodgkin's disease, USA | A | 1 |
| C. neoformans | | | |
| var. neoformans | | | |
| AVB6 | AIDS patient, The Netherlands | D | 2 |
| CBS 132 | Institut Pasteur, Paris, France | AD | 2 |
| CBS 888 | Unknown | D | 2 |
| CBS 918 | Dead white mouse | D | 2 |
| CBS 950 | Tumor | AD | 2 |
| CBS 5728 | Nonmeningitic cellulitis & osteomyelitis, USA | D | 2 |
| CBS 6885 | Lesion on bone of man, USA | D | 2 |
| CBS 6886 | Dropping of pigeon | D | 2 |
| CBS 6900 | Genetic offspring of CBS 6885 × CBS 7000 | D | 2 |
| CBS 6901 | Genetic offspring of CBS 6885 × CBS 7000 | D | 2 |
| CBS 6995 | Cerebrospinal fluid non AIDS patient, USA | D | 2 |
| CBS 7815 | Pigeon droppings, Czechoslovakia | D | 2 |
| CBS 7816 | Cuckoo dropping, Thailand | D | 2 |
| CBS 7824 | Unknown | D | 2 |
| CBS 7825 | Unknown | AD | 2 |
| PCC09 | Rio de Janeiro | D | 2 |
| RV 52755 | Cerebrospinal fluid, Belgium | D | 2 |
| WM 628 | Human, cerebrospinal fluid, Australia | AD | 2 |
| WM 629 | Human, blood, Australia | D | 2 |
| J9 | AIDS patient, USA | D | 2 |
| C. gattii | | | |
| CBS 1930 | Sick goat, Aruba | B | 3 |
| CBS 5758 | Unknown | C | 5 |
| CBS 6289 | Subculture of type strain RV 20186 | B | 4 |
| CBS 6955 | Spinal fluid, of Filobasidiella bacillispora, USA | C | 5 |
| CBS 6994 | Cerebrospinal fluid, USA | C | 5 |
| CBS 7523 | Eucalyptus camaldulensis, Australia | B | 4 |
| CBS 7748 | Air in hollow, Eucalyptus camaldulensis, Australia | B | 4 |
| CBS 7749 | Eucalyptus camaldulensis, Australia | B | 4 |
| NIH 139 | Patient, USA | C | 5 |
| NIH 178 | Patient, USA | C | 5 |
| IMH 1658 | Nest of Wasp, Uruguay | B | 3 |
| CGBMA6 | Pink shower tree, Brazil | B | 3 |
| CGBMA15 | Pink shower tree, Brazil | B | 3 |
| WM 178 | Human, lung, Australia | B | 3 |
| WM 179 | Human, cerebro spinal fluid, Australia | B | 4 |
| WM 717 | Woody debris of Eucalyptus terricornis, USA | B | 4 |
| WM 718 | Woody debris of Eucalyptus terricornis, USA | B | 4 |
| WM 726 | Eucalyptus citriodora, USA | B | 5 |
| WM 779 | Cheetah, South Africa | C | 6 |
| B5742 | Human, cerebro spinal fluid, India | C | 6 |
| B5748 | HIV patient, India | B | 6 |

CBS, Centraalbureau voor Schimmelcultures;
RV, Institute of Tropical Medicine;
NIH, National Institutes of Health;
WM, University of Sydney at Westmead Hospital (Australia).
The rest of the isolates were provided by individual researchers from America and Europe.

DNA Isolation and PCR Reaction

PCR amplifications employed either isolated DNA from cultured cells or direct detection from cells. Isolated DNA was obtained from cultured cells as described by Fell et al. (2000) using lysing enzyme and QIAmp Tissue kit (QIAGEN Inc) or by the CTAB method (62). Direct detection from cultured cells employed a pinhead size portion of a colony diluted in 15 μl of sterile, distilled water. The culture was grown for two days in GPY at 25° C. The microcentrifuge tube was vortexed, after which 4 μl of the cell suspension was transferred into the PCR reaction.

Amplification reactions used the forward primer IG1F (5′CAG ACGACTTGAATGGGAACG, SEQ ID NO:9), located at position 3613-3633 of the LrRNA region) and the reverse primer, IG2R (5′ATG CAT AGA AAG CTG TTG G, SEQ ID NO: 10) located at position no. 791 of the IGS1 region. The reverse primer was biotinylated at the 5′end. The PCR reaction was carried out in microtubes using Qiagen HotStarTaq Master Mix (QIAGEN Inc) in a final volume of 50 μl. The master mix contained: 10 ng to 1 pg of genomic DNA, 1.5 mM MgCl₂, 0.4 μM of forward and reverse primer pairs, 2.5 units of HotStarTaq polymerase, dNTPs containing 200 μM each of dGTP, dCTP, dTTP and dATP. PCR reaction employed a MJ Research PTC 100 thermocycler consisting of an initial activation at 95° C. for 15 min, followed by 35 cycles amplification: 30 sec of denaturating at 95° C., 30 sec annealing at 50° C. and 30 sec extension at 72° C. A final elongation step was applied at 72° C. for 7 min.

Capture Probe Design and Validation

Probe design for C. neoformans species complex and their genotypes employed sequence data from the IGS I region (20). These data, which are available on Gen Bank, contained over 100 sequences from clinical and environmental strains (20). Sequences were aligned with Megalign Program (DNAStar) to determine unique sequences that could be used for probe development. When possible, probes were designed to be uniform in length (21 mer). However, to avoid potential secondary structures (stem loops) or unstable delta G, some probes underwent length modification. To assess the quality of the probe, the software program Oligo™ (Molecular Biology Insights Inc.) was employed. The specificity of the prospective probe was screened with GeneBank BLAST. The secondary phase of the probe validation was achieved by testing the performance of the probe on a bead-based hybridization assay format. The capture probes, which were complementary in sequence to the biotinylated strand of the target amplicon, were synthesized with a 5′end Amino C12 modification (IDT—Coralville, Iowa). Each probe was covalently coupled to a different set of 5.6 μm polystyrene carboxylated microspheres using a carbodiimide method (32) with slight modifications (19). Coupling optimization was carried out by adjusting the amount of probe in a range of 0.2 to 0.5 nmol.

Hybridization Assay

This bead suspension assay is based upon detection of 5′biotin-labeled PCR amplicons hybridized to specific capture probes covalently bound to the carboxylated surface of the microspheres. Hybridization was performed in 3M TMAC (tetramethyl ammonium chloride/50 mM Tris, pH 8.0/4 mM EDTA, pH 8.0/0.1% sarkosyl) solution. Duplicate samples containing 5 μl of biotinylated amplicon were diluted in 12 μl of 1×TE buffer (pH 8) and 33 μl of 1.5×TMAC solution containing a bead mixture of ˜5000 microspheres of each set of probes. Prior to hybridization, the reaction mixture was incubated for 5 min at 95° C. with a PTC-100 Thermocycler (MJ Research). This step was followed by 15 min incubation at 55° C. After hybridization, the beads were centrifuged at 2250 rpm for 3 min. Once the supernatant was carefully removed, the 96 well plate was incubated for 5 min at 55° C. and the hybridized amplicons were labeled for 5 min at 55° C. with 300 ng of freshly made streptavidin-R-phycoerythrin. The samples were centrifuged and the supernatant removed. This step was followed by the addition of 75 μl of 1×TMAC. The samples were analyzed on the Luminex 100 analyzer. One hundred microspheres of each set were analyzed, which represents 100 replicate measurements. Median Fluorescent Intensity (MFI) values were calculated with a digital signal processor and the Luminex 1.7 proprietary software. Each assay was run twice. A blank and a set of positive and negative controls were included in the assay. The signal to background ratio represents the MFI signals of positive controls versus the background fluorescence of samples containing all components except the amplicon target. A positive signal corresponds to a signal, which is twice the background level, once the background has been substracted.

The sensitivity of the assay was determined with serial dilutions of genomic DNA (10 ng to 1×10⁻³ ng) and amplicons (500 to 1×10⁻³ ng). DNA quantification was determined with NanoDrop® ND-1000 spectrophotometer using an absorbance of 260 nm. Prior to quantification, amplicons were purified with Qiagen Quick-spin (QIAGEN Inc). Reactions were performed in duplicate and the experiment run twice.

To test the detection of multiple targets in a single reaction, amplicons, which were generated by a mix of genomic DNA isolates representing genotypes 1 to 5, were tested in the hybridization assay format. In order to determine the optimum parameters for multi-template PCR, several reactions were conducted using various concentrations of genomic DNA (5-10 ng); MgCl₂ (1.5-2.25 mM); dNTPs (200-300 μM); polymerase (2.5-3.75 Units) and PCR primers (0.4 to 0.8 μM). The PCR reactions were run with the standard PCR program. Five or 15 μl of amplicon was used in the hybridization assay.

To test the multiplex capability of the assay, individual sets of probes were pooled into a bead mix and tested in one and 8-plex formats. Each plex assay was tested with amplicons derived from single strains.

### EXAMPLE 1

Probe Specificity

Eight probes were designed to target the varieties and genotypic groups of the C. neoformans species complex. The probes were tested and validated with ˜66 clinical and environmental isolates listed in Tables 1 and 3. The probes were designed to have a GC content higher than 30%, Tm higher than 50° C. and a length of 21 bases. Some of the designed probes did not follow the above parameters. For example, CNG 5b displays a Tm of 48.5° C. and CNN 1b is a 20 mer oligo. All probes were coupled at 0.2 nmol, except for CNG 5b, which used 0.5 nmol. The probe sequences are depicted in Table 2.

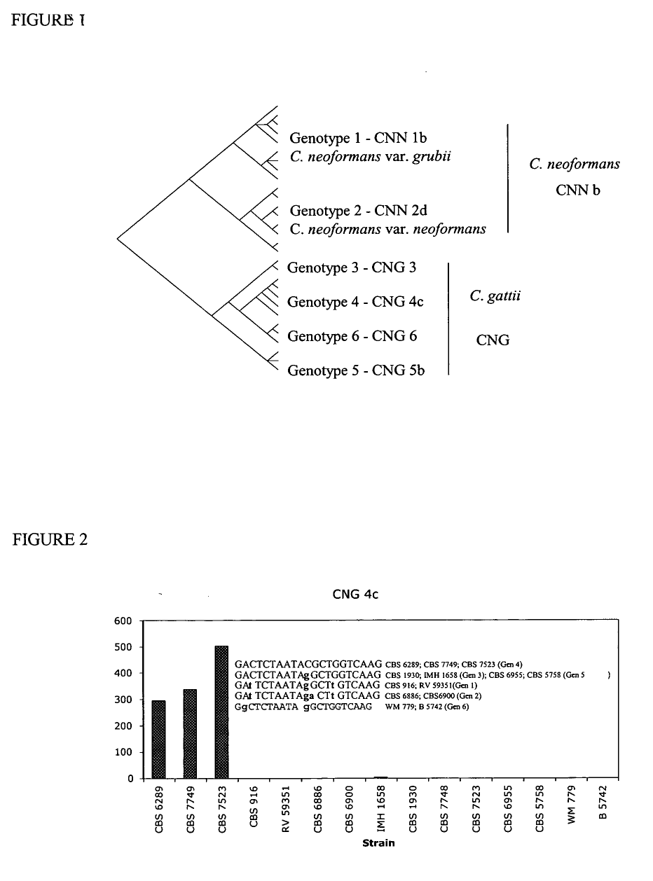

The specificity of each probe was tested against the positive control (perfect match), negative controls (more than three mismatches) and cross-reactive groups (one to three mistmatches). Six probes, represented by CNN 1b (genotype1); CNN 2d (genotype 2); CNG 3 (genotype 3); CNG 4c (genotype 4); CNG 5b (genotype 5) and CNG 6 (genotype 6) were developed to identify the genotypic groups as described in the IGS phylogenetic tree of C. neoformans species complex (FIG. 1). In addition, two group-specific probes were designed to identify members of the two main clades, represented by CNN b, which includes strains belonging to C. neoformans var neoformans/C. neoformans var. grubii and CNG, which includes all the genotypic groups (3-4-5-6) within C. gattii (FIG. 1).

The results demonstrated that under our capture assay conditions we can discriminate between probe sequences that differ by one base-pair from the target sequence. To illustrate the specificity of our assay, probe CNG 4c, which targets genotype 4 isolates, was challenged against strains belonging to different genotypic groups (FIG. 2). None of the potential cross-reactive strains, represented as those isolates displaying 1 to 3 bp differences, were found to cross-react with CNG 4c, indicating the specificity of the assay (FIG. 2).

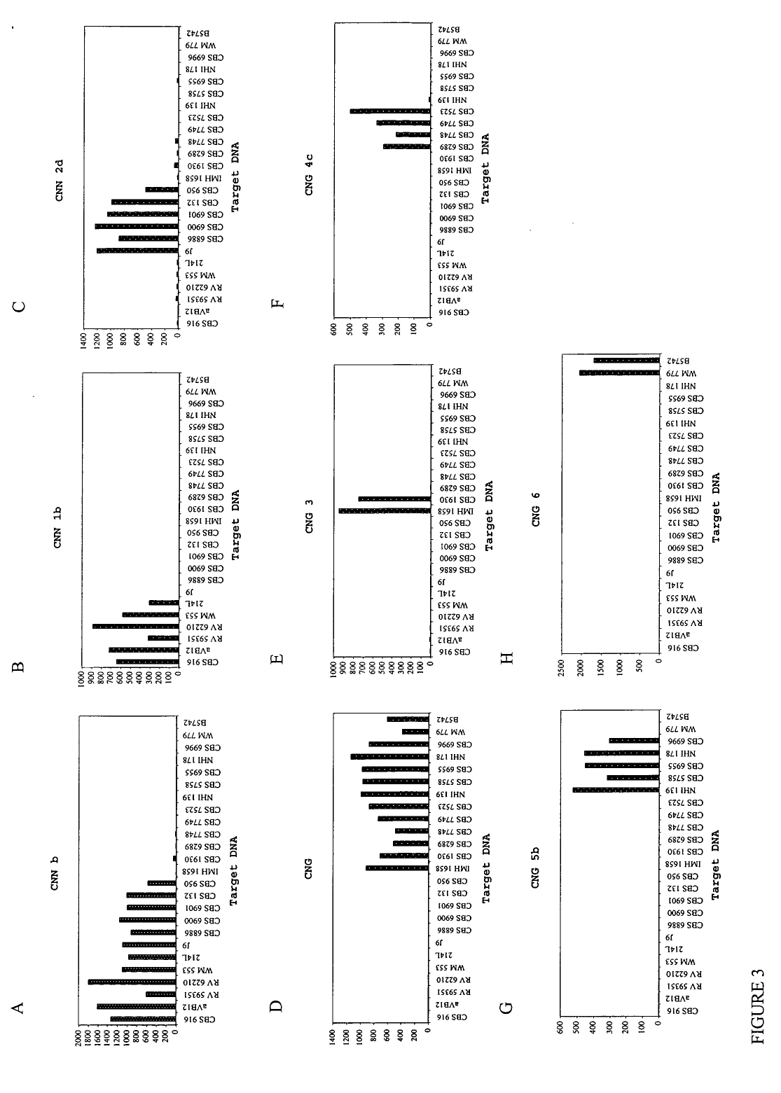

FIG. 3 A-H depicts the performance of all eight probes tested against strains representing all six genotypic groups. The probe specificity was accurate as no cross-reactivity was observed with non-target isolates. For example, CNG 6 was specific and only hybridized with perfectly matching complementary sequences of strains, e.g. WM 779 and B 5742 (FIG. 3H). No cross-hybridization was documented with non-target strains (e.g., IMH 1658, CBS 1930, CBS 6289, CBS 7748, CBS 7749, CBS 7523, NHI 139 CBS 5758 CBS 6955 and NHI 178) with two mismatches from CNG 6 probe sequence: TAAcTTCTCgCGCCCACTGTG (SEQ ID NO: 11) (FIG. 3H). Overall, the specificity of this bead-based assay was maintained when the basepair difference(s) were centrally located. An exception to this rule was CNG 5b, which maintained specificity when tested with genotype 3 isolates (IMH 1658 and CBS 1930) bearing 2 off-centered bp differences at positions 5 and 6 from the 5′end: (AAAAtgGGTAAATGTGGTATG, SEQ ID NO: 12) (FIG. 3G).

Some inherent variability in probe hybridization signal was found among positive control strains when challenged with their probe targets (FIG. 3A-H). When CNN b was tested with various genotype 1 isolates, the MFI signals for RV 62210 and CBS 950 ranged from 1800 to 576 MFI, respectively (FIG. 3A). A similar scenario, where different positive control strains displayed different signal intensities, was observed for other probes (FIG. 3 B-H). Despite the difference in signals among the positive control strains, all isolates displayed MFI values of sufficient strength to allow differentiation of positive from negative samples. In addition to the differential hybridization response among strains with complementary target sequences, we found that fluorescent intensities among probes varied considerably. For example, CNG 5b and CNG 4c displayed fluorescent signals ranging ˜250 to 500 MFI, whereas others CNN b, CNN 2d, CNG and CNG 6 displayed MFI values of over 1000.

### EXAMPLE 2

Probe Multiplexing

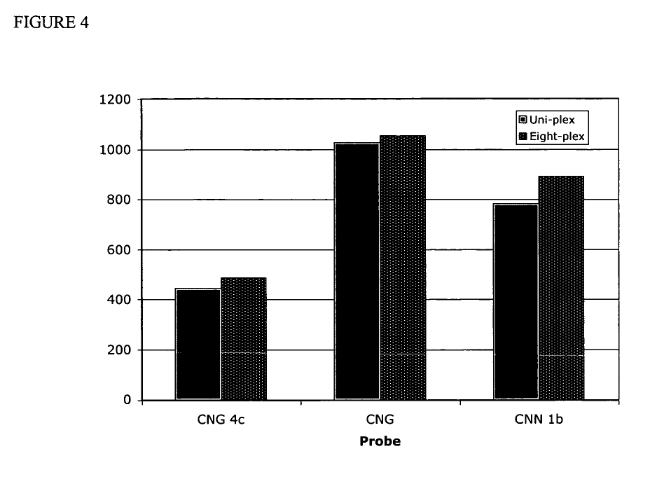

Experiments were designed to test the multiplex capability of the assay employing multiple probes in a single reaction. After the probes were pooled they were challenged with a single amplicon target per well. The results showed that all probes performed similarly when tested in uni-plex and eight-plex format. For example, FIG. 4 shows that the signal intensity of probes CNG 4c, CNG, and CNN 1b were not dramatically different when the probes were tested in both plex formats as the fluorescent signals of the uni-plex vs the eight-plex format differ by only 8, 2.7 and 12%, respectively.

### EXAMPLE 3

Probe Validation with Blind Test Isolates Derived From Clinical and Environmental Sources

Probe validation was undertaken with a blind collection of isolated DNA comprised of 16 clinical and environmental strains. Fourteen samples were clinical isolates from HIV positive individuals recovered from various hospitals in Portugal, except for CN 79, which originated from Institute Pasteur in Paris. Two strains, PYCC 5025 and CN 112 were recovered from environmental sources. Table 3 describes the source of isolation, serotype, and origin for each of the isolates, which were disclosed after conducting the blind testing. Employing our multiplex assay format, we determined without ambiguity, the varietal status and genotypic classification for each of the strains (Table 3). The varietal classification was in agreement to those submitted by the donors, who used an array of morphological, biochemical and PCR molecular techniques to identify the isolates (Dr. I. Spencer-Martins, personal communication). Among the studied strains, all twelve serotype A isolates belonged to C. neoformans var. grubii, genotype 1 (CN4, CN 32, CN 43, CN 50, CN55, CN 59, CN 70, CN 95, CN83, CN 112, CN 92, CN 74), followed by three strains (serotype AD: CN 38; CN 40; serotype D strain: CN 79), identified as C. neoformans var. neoformans genotype 2 (Table 3). The remaining isolate, (serotype B: PYCC 5025) belonged to C. gattii, genotype 4 (Table 3).

TABLE 3
Results on the identification of clinical and environmental strains used for the identification of the
genotypes and varieties of Cryptococcus neoformans species complex.

| Strains | Source | Serotype | Geographic Origin | Species | Genotype |
|---|---|---|---|---|---|
| CN 4 | CSF | A | Guine (Bissau) | C. n. var. grubii | 1 |
| CN 32 | blood | A | Hospital Sta. Maria (Lisbon) | C. n. var. grubii | 1 |
| CN 38 | blood | AD | Hospital Sta. Maria (Lisbon) | C. n. var. neoformans | 2 |
| CN 40 | CSF | AD | Hospital Sta. Maria (Lisbon) | C. n. var. neoformans | 2 |
| CN 43 | CSF | A | Inst. for Tropical Medicine (Lisbon) | C. n. var. grubii | 1 |
| CN 50 | CSF | A | Hospital Sta. Maria (Lisbon) | C. n. var. grubii | 1 |
| CN 55 | CSF | A | Hospital Sto. Antonio (Oporto) | C. n. var. grubii | 1 |
| CN 59 | CSF | A | Hospital Sta. Maria (Lisbon) | C. n. var. grubii | 1 |
| CN 70 | CSF | A | Hospital Sta. Maria (Lisbon) | C. n. var. grubii | 1 |
| CN 74 | CSF | A | Hospital Sta. Maria (Lisbon) | C. n. var. grubii | 1 |
| CN 79 | CSF | D | Institute Pasteur (Paris) | C. n. var. neoformans | 2 |
| CN 83 | CSF | A | Hospital Sta. Maria (Lisbon) | C. n. var. grubii | 1 |
| CN 92 | CSF | A | Hospital Sta. Maria (Lisbon) | C. n. var. grubii | 1 |
| CN 95 | CSF | A | Prisonal Hosp. (Lisbon) | C. n. var. grubii | 1 |
| CN 112 | pigeon droppings | A | Veterinary School (Lisbon) | C. n. var. grubii | 1 |
| PYCC 5025 | eucalyptus tree | B | Australia | C. n. gattii | 4 |

### EXAMPLE 4

Multi-Target Detection

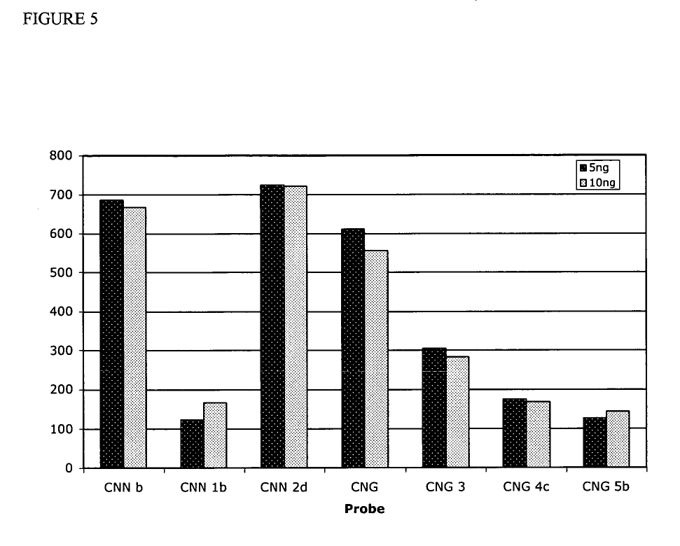

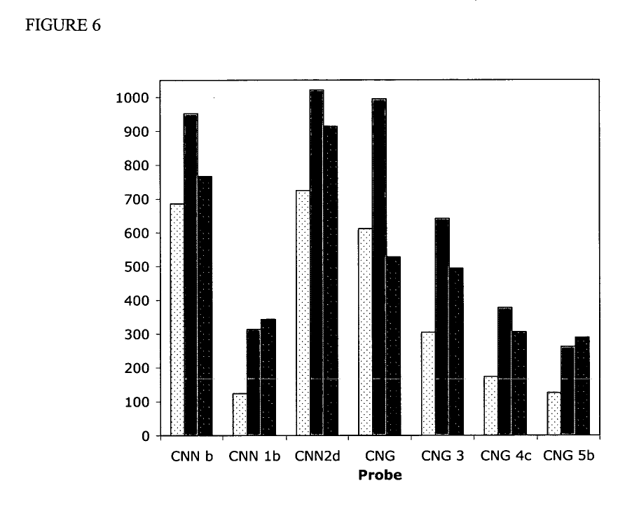

In order to determine the feasibility of xMAP to identify multiple strains in a single sample, a multi-template PCR reaction was carried out with the following genomic DNAs: WM 554 (genotype 1); CBS 132 (genotype 2); CGBMA6 (genotype 3); CBS 7523 (genotype 4) and CBS 6955 (genotype 5). These amplifications used 1.5 mM MgCl₂, 200 μM dNTPs, 2.5 units of polymerase and equimolar concentrations (0.6 μM) of the primer set, IG1F and IG2R. PCR reactions were tested with 5 and 10 ng of genomic DNA from each of the strains (FIG. 5). (Seven probes were tested against a mixture of strains representing 5 genotypes.) The generated multi-target amplicon was hybridized with the probes in a multiplexed format. Our results show that 5 or 10 ng of genomic template in the PCR reaction enabled the detection of the above isolates (CGBMA6). Overall, the sensitivity of the multiple genomic PCR was lower than the single genomic PCR reactions (FIG. 6). However, the fluorescent signal can be improved by increasing the amount of amplicon in the hybridization reaction (FIG. 6). When 15 μl of the amplicon target was used, the hybridization signals were comparable to those results with single target amplicons (FIG. 6).

### EXAMPLE 5

Genomic and Amplicon Detection Limits

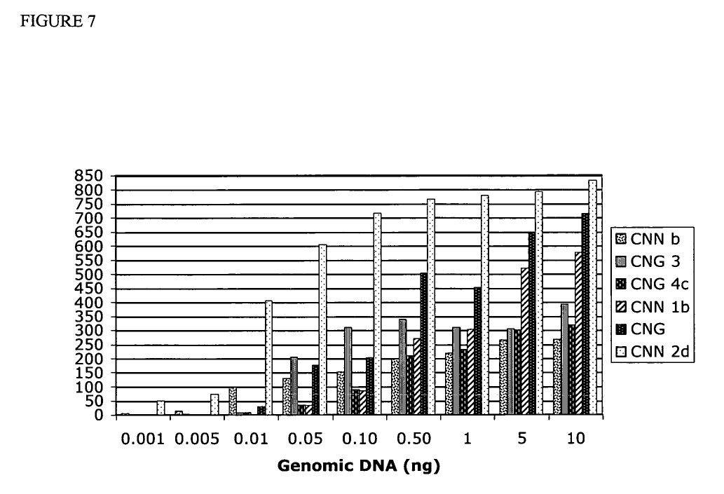

To determine the minimum amount of detectable genomic DNA in the PCR reactions, serial dilutions of genomic DNA ranging from 10 to 10⁻³ ng were performed with CNN b, CNN 1b, CNN 2d, CNG, CNG 3 and CNG 4c. The lowest limit of detection was 1 pg (CNN 2d), followed by 10 pg (CNN b and CNG). Other probes, CNN 1b, CNG 4c and CNG 3 showed detection limits of ˜50 pg (FIG. 7). Below 10 pg levels, the signal was barely detectable, except for CNN 2d, which showed detection limits as low as 1 pg of DNA with a signal intensity ˜50 MFI (FIG. 7).

Detection limits of the amplicon targets were carried out with cleaned PCR products serially diluted from 500 to 10⁻³ ng. The amplicon detection limits as determined by CNG and CNN 1b, demonstrate that this assay can detect 0.5 ng with signal intensities over 50 MFI (data not shown).

### EXAMPLE 6

Direct Detection From Cultures

Direct yeast cell amplification, which was performed with a pinhead size portion of a colony diluted in 15 μl of sterile water, demonstrated that 4 μl of the cell suspension is sufficient to generate an amplicon that can be used for the identification of the isolates without DNA extraction. For this particular experiment, we used a set of reference strains (Table 1) that had been typed by PCR fingerprinting and URA 5 RFLP (57).

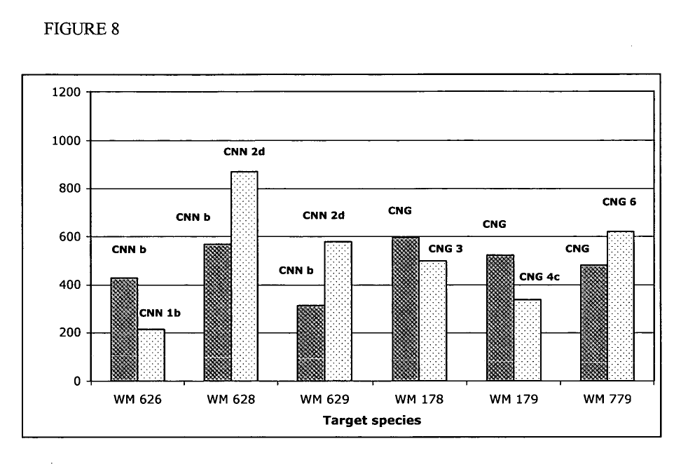

As shown in FIG. 8 we identified all six strains at variety and genotypic level by direct detection with fluorescent signals ranging from 210 to 867 MFI. The identity of the strains at genotypic level was as follows: WM 628 & WM 629: genotype 2; WM 626: genotype 1; WM 178: genotype 3; WM 179: genotype 4 and WM 779: genotype 6. For some probes, e.g. CNG and CNG 6, the MFI values obtained from direct amplification (i.e., WM 779), were reduced by 42-52% when compared to those of DNA extracted material (data not shown). Nevertheless, the displayed signal intensities of the probes with non-extracted cells ranged from ˜10 to 25 fold above background levels. The reduction in signal is probably due to differential amplification efficiencies from both techniques, which resulted in different concentrations of PCR product. For instance, the PCR concentration by direct amplification (i.e., WM 779) averaged ˜33 ng/μl as opposed to ˜50 ng/μl with extracted DNA. By increasing the amount of amplicon to 15 μl in the hybridization assay, the probe signals from non-extracted cells were enhanced by nearly 50% and were similar to those of DNA extracted cells (data not shown).

Varietal and genotypic identification of C. neoformans species complex can be of paramount importance for a correct diagnosis and an adequate selection of antifungal agent since differences in azole drug susceptibility have been reported between the varieties of C. neoformans (10). PCR molecular-based methods, e.g., reverse cross blot hybridization (70), nested-real time PCR (6) and Multiplex PCR (13, 55) have been applied successfully for the identification of C. neoformans in clinical specimens. However, none of these methods can identify the species at the variety or genotypic level.

Herein, we successfully adapted Luminex xMAP technology to differentiate between the varieties and genotypes of one of the most important fungal pathogens, C. neoformans. Differences in the non-conservative region of the rRNA gene, IGS region, allowed us to develop and validate eight different probes that can target the varieties and the different molecular genotypes of the species. This technique, which incorporates flow cytometry and a bead based captured hybridization assay, was a reliable method for the detection of C. neoformans species complex. A similar assay has been successfully employed for the identification of all species within the genus, Trichosporon (19, and U.S. patent application Ser. No. 11/134,619, filed May 23, 2005).

In conventional hybridization assays, discrimination between perfect match and single-basepair mismatched duplexes is generally achieved by controlling the temperature, ionic strength, inclusion of formamide or by the addition of stringent washes with low salt concentration (56). Another strategy is to analyze the melting profiles of individual probes spotted on a chip surface (52). Under the present hybridization assay format, which involved a short incubation at 55° C., we were able to meet the stringent conditions necessary to discriminate among sequences with 1 bp mismatches by the inclusion of 3M TMAC. This quarternary alkylammonium salt eliminates the preferential melting of AT vs. GC base pairs, allowing multiple probes with different base pair composition to be employed under similar hybridization conditions (80). An example of the specificity of the assay is illustrated in FIG. 2, where no cross-reactivity was observed among isolates bearing one mismatch from the probe sequence. Similar specificity was attained in our previous study employing a similar hybridization assay for the detection of Trichosporon spp. (19). As observed, the specificity of the probe was maintained if the mismatches are located at positions 9 through 11 from the 5′ or 3′end (FIG. 2). However, if a probe sequence has two consecutive mismatches that are off-centered at positions 5 to 6 from the 5′ end, it is possible to retain the specificity. For instance, none of the strains (genotype 3) bearing two consecutive mismatches from the probe sequence of CNG 5b cross-reacted with that particular oligo. According to the kinetics of dissociation, the maximum destabilizing effect of a mismatch is achieved when the mismatches are in the center of the sequence (34) and when the mismatches involves A-A, T-T, C-T and C-A (42). Double consecutive mismatches after the last three end positions are known to produce unstable duplexes, especially if one of the mutations like those portrayed in CNG 5b, involves a C-T, which is considered a significant destabilizing mismatch (42, 50). Mismatches involving C-T can lead to a significant distortion in the helical structure due to the small size of the pyrimidine-pyrimidine base pair, which results in an unstable duplex. (42).

In the current study, some heterogeneity in hybridization signals was observed among strains belonging to the same genotypic groups. This effect has been reported by others and is manly due to differential yields in PCR products, or PCR labeling efficiencies (54), which can be associated to the quality and/or concentration of the genomic template. Similarly, different probes exhibited different signal intensities after hybridizing with their perfectly matched target. This wide range of fluorescence signals, which in our case ranged from ˜250-2000 MFI above background levels, has been attributed to: base composition, base stacking interaction, steric hindrance, position of the probe binding site, secondary structures of the single stranded target molecule, hairpin structures in the probe sequence, and kinetics (35, 40, 65, 76). Although all of these factors can have a profound effect on the duplex yield, and fluorescent intensity associated with probe-target match, the complex interaction of the above mechanisms remains a puzzle. In trying to elucidate this dilemma, some investigators have developed secondary structural maps of the D1/D2 of rRNA or the 23 rRNA gene to evaluate the accessibility of fluorescent probes based on secondary structural conformations of the different domains, but the results are non-conclusive (31, 35, 40).

The sensitivity of the assay as determined by the amount of genomic DNA in the PCR reaction indicated that under our assay conditions we detected between 10 and 50 pg of genomic DNA. However, for probe CNN 2d, we detected as little as 1 pg. These detection levels can be further improved by increasing the amount of amplicon in the assay as we demonstrated in a similar assay format for the detection of the pathogenic yeast, Trichosporon (19). Our detection levels are more sensitive than studies based on PCR-EIA and molecular beacon probes that report detection limits of 1 ng and 100 pg for the detection of clinically important fungi (26, 64).

Considering the genome size of C. neoformans (24 MB), we estimated that 1, 10 and 50 pg of genomic DNA template correspond to a detection limit of 38, 380 and 1,900 genome copies, respectively. When converting to cell numbers, the detection limits for C. neoformans species complex ranged from 4×10¹ to 2×10³ cells. Considering that pathogenic yeasts positive blood cultures normally exceeds 10⁵ CFU/ml (14) and the quantity of yeast in CSF specimen ranges from 10³ to 10⁷/CFU/ml (67), our detection levels should be sensitive for the detection and identification of this pathogen in clinical specimens.

The detection limits of the amplicon products, which were assessed with dilution series of the amplification products, showed that the lowest amount of product for both CNG and CNN 1b was 0.5 ng, which represent 5.81 fmol and 1.65 fmol, respectively. These detection levels are identical to those reported by Chen et al. (2000), who employed the same technology for the identification of single nucleotide polymorphism (SNPs). Diaz and Fell (2004) reported slightly less sensitive values ranging from 1 to 5 ng for the identification of Trichosporon spp. A sensitivity of 1 ng, was reported for the identification of Candida species using PCR-EIA (26). After correction for amplicon length and copy numbers, this sensitivity is equivalent to 106 amplicon copies for CNN 1b and 10⁷ for CNG. These amplicon detection levels are concordant to those reported by Dunbar et al. (24), who used the same technology for the identification of bacterial pathogens.

Direct amplification from cultures demonstrated the feasibility of performing the assay without DNA extraction. This two-day culture procedure was used to standardize the assay conditions and can be applied to cultures of any age. The successful amplification of intact cells was probably due to factors associated with sufficient content of template in the cell suspension and the high copy numbers of the target region rRNA, which in fungi are present in hundreds of copies (46, 71). The high copy number can act as a pre-amplification step, enabling an increase in amplicon yield (51, 53).

Multi-template-PCR reactions, which were carried out with 5 strains representing five different genotypic groups, demonstrated that we can detect and correctly identify multiple strains in a single sample employing the described hybridization assay format. However, to accommodate all five strains in a single PCR reaction, and minimize preferential amplification of target sequences, certain modifications involving an increase in primer concentration, DNA template and amplicon amount, were necessary to achieve successful amplification and identification. The simultaneous screening of pathogenic strains is a practical way to identify multiple species or IGS genotypes co-existing in a single host or environmental source. For instance, Lazera et al. (2000) reported the occurrence of C. neoformans var. neoformans and C. gatti in the same environmental habitat. Even though multiple infection of strains with different IGS molecular types can be a rare occurrence in specimens from single patients or environmental source, the fact that we can identify C. neoformans at species, variety or IGS genotypic level in a single sample, illustrates the potential and capability of the assay, which could be easily adapted for the simultaneous identification of other fungal pathogenic species.

In conclusion, we have adapted this high-throughput technology toward the identification of the species complex C. neoformans from culture-based material. The assay described in this study proved to be specific, sensitive, and flexible, allowing a complete array of different target species to be identified in a multiplex format by pooling probes of interest. In addition to the multiplex capability, the developed assay has the potential to identify multi-species or strains in a single sample. The assay can be executed in less than an hour after the amplification step. Although most of our experiments used extracted DNA, we demonstrated that this step could be omitted as biotinylated amplicons can be generated directly from intact yeast cells. Once the probes are developed, the cost of operation is relatively low. The above options not only decrease sample preparation time, the amount of reagent used and the amount of sample needed and reduce the cost of the assay. All of these aspects make this assay useful for applications in clinical settings, where there is a demand for a high-throughput system that allows the creation of multiple testing platforms for routine diagnostic testing.

Publications, patent applications, patents and references cited herein are hereby incorporated by reference.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### REFERENCES

1. Aguirre, L., S. F. Hurst, J. S. Choi, J. H. Shin, H. P. Hinrikson, and C. J. Morrison. 2004. Rapid differentiation of Aspergillus species from other medically important opportunistic molds and yeasts by PCR-enzyme immunoassay. J. Clin. Microbiol. 42:3495-3504.
2. Atkins, S. D., and I. A. M. Clark. 2004. Fungal molecular diagnostics: a mini-review. J. Appl. Genet. 45:3-15.
3. Banerjee U., K. Datta, and A. Casadevall. 2004. Serotype distribution of Cryptococcus neoformans in a tertiary care center in India. Med Mycol. 42:181-186
4. Bava, A. J., R. Negroni, and M. Bianchi. 1993. Cryptococcosis by a urease negative strain of Cryptococcus neoformans. J. Med. Vety. Mycol. 31:87-89.
5. Bennett, J. E., K. J. Kwon-Chung, and D. Howard. 1977. Epidemiologic differences among serotypes of Cryptococcus neoformans. Am. J. Epidemiol. 105:582-586.
6. Bialek, R., M. Weiss, K. Bekure-Nemariam, L K. Najvar, M. B. Alberdi, J. R. Graybill and U. Reischl. 2002. Cryptococcus neoformans DNA in Tissue Samples by Nested and Real-Time PCR Assay. Clin. Diagn. Lab. Immunol. 9:461-469.
7. Blevins, L. B., J. Fenn, H. Segal, P. Newcomb-Gayman, and K. C. Carroll. 1995. False-positive cryptococcal antigen latex agglutination cused by disinfectants and soaps. J. Clin. Microbiol. 33:1674-1675.
8. Boekhout, T., B. Theelen, M. Diaz, J. W. Fell, C. J. Hop, E. C. A. Abeln, F. Drommer, and W. Meyer. 2001. Hybrid genotypes in the pathogenic yeast Cryptococcus neoformans. Microbiol. 147:891-907.
9. Boom, W. H., D. J. Piper, K. L. Rouff, M. J. Ferrano. 1985. New cause for false positive results with cryptococcal antigen test by latex agglutination. J. Clin. Microbiol. 22:856-857.
10. Brummer, E., K. Kamei and M. Miyaji. 1998. Anticryptococcal activity of voriconazole against Cryptococcus neoformans var. gatti vs neoformans: Comparison with fluconazole and effect of human serum. Mycopathologia 142:3-7.
11. Calvo, B. M., A. L. Combo, O. Fischman, A. S. Santiago, L. Thompson, M. Lazera, F. Telles, K. Fukushima, K. Nishimura, R. Tanaka, M. Myajy, and L. Morettii-Branchini. 2001. Antifungal susceptibilities, varieties, and electrophoretic karyotipes of clinical isolates of Cryptococcus neoformans from Brazil, Chile and Venezuela. J. Clin. Microbiol. 30:2348-2350.
12. Casadevall, A., and J. R. Perfect. 1998. Cryptococcus neoformans. ASM Press, Washington D.C., USA, pp. 1-541.
13. Chang, H. C., S. N. Leaw, A. H. Huang, T. L. Wu, and T. C. Chang. 2001. Rapid identification of yeasts in positive blood cultures by a multiplex PCR method. J. Clin. Microbiol. 39:3466-3471.
14. Chang, H. C., J. J. Chang, A. H. Huang, and T. C. Chang. 2000. Evaluation of a capacitance method for direct antifungal susceptibility testings of yeasts in positive cultures. J. Clin. Microbiol. 38:971-976.
15. Chen J., M. A. Iannone, M. S. Li, J. D. Taylor, P. Rivers, A. J. Nelsen, K. A. Slentz-Kesler, A. Roses and M. P. Weiner 2000. A microsphere-based assay for multiplexed single nucleotide polymorphism analysis using single base chain extension. Genome Res. 10:549-556.
16. Cogliati, M, M. Allaria, A. M. Tortorano, and M. A. Viviani. 2000. Genotyping Cryptococcus neoformans var neoformans with specific primers designed from PCR-fingerprinting bands sequenced using a modified PCR-based strategy. Med. Mycol. 38:97-103.
17. Correa, M. P. S. C., L. C. Severo, and F. M. Oliveira. 2002. The spectrum of computerized tomography (CT) findings in central nervous system (CNS) infection due to Cryptococcus neoformans var. gattii in immuno-competent children. Rev. Inst. Trop. S Paulo, 44:283-287.
18. Darzé, C., R. Lucena, I. Gomes, and A. Melo. 2000. Caracteristicas clinicas laboratoriais de 104 casos de meningoencefalite criptocócica. Rev. Soc. Bras. Med. Trop. 33:21-26.
19. Diaz, M. R., and J. W. Fell. 2004. High-throughput detection of pathogenic yeasts in the genus Trichosporon. J. Clin. Microbiol. 42:3696-3706
20. Diaz M. R., T. Boekhout, B. Theelen, J. W. Fell. 2000. Molecular sequence analyses of the Intergenic Spacer (IGS) associated with rDNA of the two varieties of the pathogenic yeast, Cryptococcus neoformans. System. Appl. Microbiol. 23:535-545.
21. Diaz M. R., and J. W. Fell. 2000. Molecular analyses of the IGS and ITS regions rDNA of the psychrophilic yeasts in the genus Mrakia. Antonie van Leeuwenhoek 77:7-12
22. Dromer F., S. Mathoulin-Pelissier, A. Fontanet, O. Ronin, B. Dupont, O. Lortholary, French Cryptococcosis Study Group. 2004. Epidemiology of HIV-associated cryptococcosis in France (1985-2001): comparison of the pre and post-HAART eras. AIDS. 18:555-562.
23. Dromer, F., A Varma, O. Ronin, S. Mathhoulin, and B. Dupont. 1994. Molecular typing of Cryptococcus neoformans var. neoformans serotype D clinical isolates. J. Clin. Microbiol. 32:2364-2371.
24. Dunbar, S. A., C. A. Vander Zee, K. G. Oliver, K. L. Karem and J. W. Jacobson. 2003. Quantitative, multiplexed detection of bacterial pathogens: DNA and protein applications of the Luminex LabMap system. J. Microbiol. Meth. 53:245-252.
25. Dupont, B., H. H. Crewe Brown, K. Westermann, M. D. Martins, J. H. Rex, O. Lortholary, and C. A. Kauffmann. 2000. Mycoses in AIDS. Med Mycol. 38:259-267.
26. Elie, C. M., T. J. Lott, E. Reiss, and C. J. Morrison. 1998. Rapid identification of Candida species with species-specific probes. J. Clin. Microbiol. 36:3260-3265.
27. Ellis, D., D. Marriott, R. A. Haijjeh, D. Warnock, W. Meyer, and R. Barton. 2000. Epidemiology:surveillance of fungal infections. Med. Mycol. 38:173-182.
28. Ellis, D. H., and T. J. Pfeiffer. 1990. Natural habitat of Cryptococcus neoformans var. gatti. J. Clin. Microbiol. 28:1642-1644.
29. Fell, J. W., T. Boekhout, A. Fonseca, G. Scorzetti, and A. Statzell-Tallman. 2000. Biodiversity and systematics of basidiomycetous yeasts as determined by large subunit rDNA D1/D2 domain sequence analysis. Int. J. Syst. Bact. 50:1351-1371.
30. Fell, J., and G. Blatt. 1999. Separation of strains of the yeasts Xanthophyllomyces dendrorhousand Phaffia rhodozyma based on rDNA IGS and ITS sequence analysis. J Ind Microbiol Biotechnol. 23:677-681
31. Fuchs, B. E., K. Syutsubo, L. Wolfgang, and R. Amann. 2001. In situ accessibility of Escherichia coli to 23S rRNA to Fluorescently labeled oligonucleotide probes. Appl. Environ. Microbiol. 67:961-968.
32. Fulton R, R McDade, P. Smith, L. Kienker, and J. Kettman. 1997. Advanced multiplexed analysis with the FlowMetrix system. Clin. Chem 43:1749-1756.
33. Goldman, D. L, H. Khine, J. Abadi, D. J. Lindenberg, L. Pirofski, R. Niang, and A. Casadevall. 2001. Serologic evidence for Cryptococcus neoformans Infection in early childhood. Pediatrics. 107:66.
34. Gotoh, M., Y. Hasegawa, Y. Shinohara, M. Schimizu, and M. Tosu. 1995. A new approach to determine the effect of mismatches on kinetic parameters in DNA hybridization using an optical biosensor. DNA Res. 2:285-293.
35. Graves J. D. 1999. Powerful tools for genetic analysis come to age. Tib Tech. 17:127-134.
36. Heelan, J. S., L. Corpus, and N. Kessimian. 1991. False positive reactions in the latex agglutination test for Cryptococcus neoformans antigen. J. Clin. Microbiol. 29:1260-1261.
37. Hendolin, P. H., L. Paulin, P. Koukila-Kahkola, V. J. Anttila, H. Malmberg, M. Richardson, and J. Ylikoski. 2000. Panfungal PCR and multiplex liquid hybridization for detection of fungi in tissue specimens. J. Clin. Microbiol. 38:4186-4192.
38. Husain, S., M. Wagener, and A Singh. 2001. Cryptococcus infection in organ transplant recipients: variables influencing clinical characteristics and outcome. Emerg. Infect. Dis. 7:375-381.
39. Hsu, M. C., K. W. Chen, H. J. Lo, Y. C. Chen, M. H. Liao, Y. H. Lin, and S. Y. Li. 2003. Species identification of medically important fungi by use or real time light cycler PCR. J. Med. Microbiol. 52:1071-1076.
40. Inacio, J., S. Behrens, B. M. Fuchs, A. Fonseca, I. Spencer-Martins, and R. Amann. 2003. In situ accessibility of Saccharomyces cerevisiae 26S rRNA to Cy3-labeled oligonucleotide probes comprising the D1 and D2 domains. Appl. Environ. Microbiol. 69:2899-2905.
41. Jaeger, E. E. M., N. M. Carrol, S. Choudhury, A. A. S. Dunlop, H. M. A. Towler, M. M. Matheson, P. Adamson, N. Okhravi, and S. Lightman. 2000. Rapid detection and identification of Candida, Aspergillus, and Fusarium species in ocular samples using nested PCR. J. Clin. Microbiol. 38:2902-2908.
42. Ikuta S., K. Takagi, RB Wallace, and K. Ikura. 1987. Dissociation kinetics of 19 base paired oligonucleotide-DNA duplexes containing single mismatched base pairs. Nucl. Acid. Research. 15:797-811.
43. Katsu, M, S. Kidd, A. Ando, M. L. Morettii-Branchini, Y. Mikami, K. Nishimura, and W. Meyer. 2004. The internal transcribed spacers and 5.8 srRNA gene show extensive diversity among isolates of Cryptococcus neoformans species complex. FEMS Yeast Res. 4:377-388.
44. Kidd, S. E., F. Hagen, R. L. Tscharke, M. Huynh, K. H. Barlett, M. Fyfe, L. Macdougall, T. Boekhout, K. J. Kwong-Chung, and W. Meyer. 2004. A rare genotype of Cryptococcus gattii caused the cryptococcosis outbreak on Vancouver Island (British Columbia, Canada). Proc Natl. Acad Sci USA 101:17258-17263.
45. Kovacs, J. A., A. A. Kovacs, M. Polis, W. C. Wright, V. J. Gill, C. U. Tuazon, E. P. Gelmann, H. C. Lane, R. Longfield, G. Overturf, A. M. Macher, A. S. Fauci, J. E. Parrillo, J. E. Bennett, and H. Masur. 1985. Cryptococcosis in the acquired immunodeficiency syndrome. Ann. Intern. Med. 103:533-538.
46. Kovayashi, T., D. J. Heck, M. Nomura, and T. Horiuchi. 1998. Expansion and contraction of ribosomal DNA repeats in Saccharomyces cerevisiae: requirement of replication fork blocking (Fob1) protein and the role of RNA polymerase I. Genes Dev. 12:3821-3830
47. Kwon-Chung, K. J., and J. E. Bennett. 1984. Epidemiologic differences between the two varieties of Cryptococcus neoformans. Am. J. Epidemiol. 120:123-130.
48. Latouche, G. N., M. Huynh, T. C. Sorrell, and W. Meyer. 2003. PCR-restriction fragment polymorphism analysis of the phospholipase B (PLBI) gene for subtyping of Cryptococcus neoformans isolates. App. Env. Microbiol. 69:2080-2085.
49. Lazera, M. S., S. Cavalcanti, M. A., A. T. Londero, L. Trilles, M. M., Nishikawa, and B. Wanke. 2000. Possible primary ecological niche of Cryptococcus neoformans. Med. Mycol 38:379-383.
50. Lee, I., A. A. Dombkowski, and B. D. Athey. 2004. Guidelines for incorporating non perfectly matched oligonucleotides into target-specific hybridization probes for DNA microarray. Nucl. Acid Res. 32:681-690.
51. Lindsley M. D., S. F. Hurst, N. J. Iqbal, and C. J. Morrison. 2001. Rapid Identification of dimorphic and yeast like fungal pathogens using specific probes. J. Clin. Microbiol. 39:3505-3511.
52. Liu, W. T., A. D. Mirzabekov, and D. A. Stahl. 2001. Optimization of an oligonucleotide microchip for microbial identification studies:a non equilibrium dissociation approach. Environ. Microbiol 3:619-629.
53. Lott, T. J., R. J. Kuykendall, and E. Reiss. 1993. Nucleotide sequence analysis of the 5.8S rDNA and adjacent ITS2 region of Candida albicans and related species. Yeast 9:1199-1206.
54. Loy, A, A. Lehner, N. Lee, J. Adamczyk, H. Meier, J. Ernst, K H Schleifer, and M. Wagner. 2002. Oligonucleotide microarray for 16S rRNA gene-based detection of all recognized lineages of sulfate-reducing prokaryotes in the environment. Appl. Environ. Microbiol. 68:5064-5081.
55. Luo, G, and T. G. Mitchell. 2002. Rapid identification of pathogenic fungi directly from cultures by using multiplex PCR. J. Clin. Microbiol. 40:2860-2865.
56. Meinkoth, J, and G. Wahl. 1984. Hybridization of nucleic acids immobilized on solid supports. Anal. Chem. 138:267-284.
57. Meyer, W., A. Castaneda, S. Jackson, M. Huynh, and E. Castaneda. 2003. Molecular typing of IberoAmerican Cryptococcus neoformans isolates. Emerg Infect Dis. 2:189-95.
58. Meyer, W., K. Marszewska, M. Amirmostofian, R. P. Igreja, C. Hardtke, K. Methling, M. A. Viviani, A. Chindamporn, S. Sukroongreung, M. A. John, D. H. Ellis, and T. C. Sorrell. 1999. Molecular typing of global isolates of Cryptococcus neoformans by a polymerase chain reaction fingerprinting and randomly amplified polymorphic DNA: a pilot study to standardize techniques on which to base a detailed epidemiological survey. Electrophoresis 20:1790-1799.
59. Millon, L. T., M. C. Julliot, J. Martinez, G. Manton. 1995. Interference by hydroxyl starch used for vascular filling in latex agglutination test for cryptococcal antigen. J. Clin. Microbiol. 33:1917-1919.
60. Mirza, S. A, M. Phelan, D. Rimland, E. Graviss, R. Hamill, M. E. Brandt, T. Gardner, M. Sattah, G. P. de Leon, W. Baughman, and R. A. Hajjeh. 2003. The changing epidemiology of cryptococcosis: an update from population-based active surveillance in 2 large metropolitan areas, 1992-2000. Clin Infect Dis. 36:789-94.
61. Mwaba, P., J. Mwansa, C. Chintu, J. Pobee, M. Scarborough, S. Portsmouth and A. Zumla. 2001. Clinical presentation, natural history, and cumulative death rates of 230 adults with primary cryptococcal meningitis in Zambian AIDS patients treated under local conditions. Postgrad Med J. 77:769-73.
62. O'Donnell, E. Cigelnik, N. S. Weber and J. M. Trappe. 1997. Phylogenetic relationships among ascomycetous truffles and the true and false morels inferred from 18S and 28S ribosomal DNA sequence sequence analysis. Mycologia 89:48-65.
63. Pappalardo, M. C. S. M., and M. S. C. Melhem. 2003. Cryptococcosis: A review of the Brazilian experience for the disease. Rev. Inst. Med. Trop. S. Paolo 45:299-305.
64. Park, S., Wong M, A. Salvatore, E. Marras, E. W. Cross, T. E. Kiehn, V. Chaturvedi, S. Tyagi, and D. S. Perlin. 2000. Rapid Identification of Candida dubliniensis using a species specific Molecular Beacon. 38:2829-2836.
65. Peplies, J., F. O. Glockner, and R. Amann. 2003. Optimization strategies for DNA microarray-based detection of bacteria with 16sRNA-targeting oligonucleotide probes. Appl Environ. Microbiol. 69:1397-1407.
66. Perfect, J. R., N. Ketabchi, G. M. Cox, C. W. Ingram, and C. L. Beiser. 1993. Karyotyping of Cryptococcus neoformans as an epidemiological tool. J. Clin. Microbiol. 31:3305-3309.
67. Perfect, J. R., D. T. Durack, and H. A. Gallis. 1983. Cryptococcemia. Medicine 62:98-109.
68. Picken, M. M., R. N. Picken, D. Han, Y. Cheng, and F. Strle. 1996. Single-tube nested polymerase chain reaction assay based on flagellin gene sequences for detection of Borrelia burgdorferi sensu lato. Eur. J. Clin. Microbiol. Infect. Dis. 15:489-498.
69. Polanco, C., and M. Perez de la Vega. 1994. The structure of the rDNA intergenic spacer of Avena sativa L.: a comparative study. Plant Molec Biol 25: 751-756.
70. Posteraro, B., M. Sanguinetti, L. Masucci, L. Romano, G. Morace, and G. Fadda. 2000. Reverse cross blot hybridization assay for raid detection of PCR amplified from Candida species, Cryptococcus neoformans, and Saccharomyces cerevisiae in clinical samples. J. Clin. Microbiol. 38:1609-1614.
71. Prokopowich, C. D., T. R. Gregory, and T. J. Crease. 2003. The correlation between rDNA copy numbers and genome size in eukaryotes. Genome 46:48-50.
72. Rappelli, P., R. Are, G. Casu, P. L. Fiori, P. Cappuccinelli, and A. Aceti. 1998. Development of a nested PCR for detection of Cryptococcus neoformans in cerebrospinal fluid. J. Clin. Microbiol. 36:3438-3440
73. Saag M. S., G. A. Cloud, J. R. Graybill, J. D. Sobel, C. U. Tuazon, P. C. Johnson, W. J. Fessel, B. L. Moskovitz, B. Wiesinger, D. Cosmatos, L. Riser, C. Thomas, R. Hafner, W. E. Dismukes. 1999. A comparison of itraconazole versus fluconazole as maintenance therapy for AIDS-associated cryptococcal meningitis. National Institute of Allergy and Infectious Diseases Mycoses Study Group. Clin Infect Dis 30:291-296
74. Sar, B., D. Monchy, M. Vann, C. Keo, J. L. Sarthou, and Y. Buisson. 2004. Increasing in vitro resistance to fluconazole in Cryptococcus neoformans Cambodian. J. Antimicrob. chemother. 54:563-565.
75. Singh, N., S. Husain, M De vera, T. Gayowski, and T. V. Cacciarelli. 2004. Cryptococcus neoformans infection in patients with cirrhosis, including liver transplant. Medicine. 83:188-192.
76. Southern, E. K. Mir, and M. Shchepinov. 1999. Molecular interactions on microarrays. Nat. Genet. (supplement) 21:5-9.
77. Steenbergen, J. N., and A. Casadevall. 2000. Prevalence of Cryptococcus neoformans var. neoformans (serotype D) and Cryptococcus neoformans var. grubii (serotype A) isolates in New York City. J. Clin. Microbiol. 38:1974-1976.
78. Stephen C., S. Lester, W. Black, M. Fyfe, and S. Raverty. 2002. Multispecies outbreak of cryptococcosis on southern Vancouver Island, British Columbia. Can. Vet. J. 43:792-794.
79. Tanaka, K. I., T. Miyazaki, S. Maesaki, K. Mitsutake, H. Kakeya, Y. Yamamoto, K. Yanagihara, M. A. Hossain, T. Tashiro, and S. Kohno. 1996. Detection of Cryptococcus neoformans in patients with pulmonary cryptococcosis. J. Clin. Microbiol. 34:3438-3440.
80. Wood W., J. Gitschier, L. A. Lasky, and R. M. Lawn. 1985. Base pair composition—independent hybridization in tetramethylammonium chloroide: A method for oligonucleotide screening of highly complex gene libraries. Proc. Natl. Acad. Sci. USA 82:1585-1588.
81. Carlson D., J. Y. Lo, D. Ally, and E. Ubil. 2000. Microsphere assay for Y chromosome SNPs. Proceedings of Eleventh International Symposium on Human Identification, October 10-13 Mississippi.
82. Oliver, K., L. Patel, J. Kemp, J. Daves, L. Bell and, R. Zivin. 1999. The Luminex LabMAP system: a rapid, homogeneous, multianalyte platform. Society for Biomolecular Screening Meeting, Edinbugh, UK September 1999.
83. Spiro, A., M. Lowe, and D. Brown. 2000. A bead-based method for the multiplexed quantitation of DNA sequences using flow cytometry. Appl. Environ. Microbiol. 66:4258-4265.
84. Williams, J. C., S. C. Case-Green, S. C. Mir., and E. M. Southern. 1994. Studies of oligonucleotide interactions by hybridization to arrays: the influence of dangling ends on duplex yield. Nucleic. Acids Res. 22:1365-1367.
85. Ye, F., M. S. LI, J. D. Taylor, Q. Nguyen, H. M. Colton, W. M. Casey, M. Wagner, M. P. Weiner, and J. Chen. 2001. Fluorescent microsphere-based readout technology for multiplexed human single nucleotide polymorphism analysis and bacterial identification. Hum. Mutat. 17:305-316.

We claim:

1. An isolated nucleic acid sequence comprising a DNA sequence selected from Table 2, a complement thereof, or a corresponding RNA sequence.

2. A capture probe comprising a nucleic acid sequence of claim 1.

3. A composition comprising a capture probe of claim 2 that is bound to a solid support.

4. The composition of claim 3 wherein the solid support is a fluorescent bead.

5. A composition containing a plurality of capture probes as claimed in one of claims 2-4.

6. The composition of claim 5 comprising at least 5 of said capture probes.

7. A method for detecting a fungal pathogen comprising the steps of
providing at least one capture probe of claim 2;
contacting said capture probe(s) with a biological sample that may contain target species of nucleic acid for which said capture probe(s) are specific under conditions such that the target species will become bound to the probe to produce a hybridized product;
detecting the presence or absence of hybridized product, the presence of said hybridized product being indicative of the presence of said fungal pathogen.

8. The method of claim 7 that further comprises quantitating the hybridized product.

9. The method of claim 7 wherein the capture probe is bound to a solid support.

10. A method for detecting fungal pathogens comprising the steps of
obtaining a set of fluorescent beads covalently bound to capture probes;
contacting said fluorescent beads with a biological sample that may contain amplicons of target species for which said capture probes are specific under conditions such that said amplicons will become bound to the probe to produce a hybridized product;
using a first laser to classify the beads by their spectral addresses; and
detecting the presence or absence of said hybridized product, the presence of said hybridized product being indicative of the presence of said fungal pathogen.

11. The method of claim 10 further comprising the step of quantitating hybridized biotinylated amplicons using fluorescent detection.

12. The method of claim 10 wherein said first laser has a wavelength of 635 nm.

13. The method of claim 11 wherein the hybridized biotinylated amplicons are quantified with a 532 nm laser.

14. The method of claim 10, wherein the capture probe is specific for a species or strain from the genus Cryptococcus.

15. The method of claim 14 wherein the capture probe is specific for a strain of C. neoformans or C. gattii.

16. The method of claim 10 wherein the capture probes are selected from Table 2.

17. A kit comprising at least one capture probe of claim 2, 3 or 4, optionally including instructions for use.

18. The kit of claim 17 containing a plurality of capture probes as claimed in one of claims 2-4.

19. The kit of claim 18 comprising at least 5 of said capture probes.
